(19) United States
(12) Patent Application Publication
(10) Pub. No.: US 2024/0312614 A1
(43) Pub. Date: Sep. 19, 2024

(54) DISASSEMBLING ACCESSORY, AND METHOD FOR DISASSEMBLING PHYSIOLOGICAL SIGNAL MONITORING DEVICE

(71) Applicant: BIONIME CORPORATION, Taichung City (TW)

(72) Inventors: Chun-Mu HUANG, Taichung City (TW); Hsueh-Chuan CHANG, Taichung City (TW); Hung-Wen CHIANG, Taichung City (TW)

(73) Assignee: BIONIME CORPORATION, Taichung City (TW)

(21) Appl. No.: 18/605,132

(22) Filed: Mar. 14, 2024

Related U.S. Application Data
(60) Provisional application No. 63/490,314, filed on Mar. 15, 2023.

(51) Int. Cl. G16H 40/40

(52) U.S. Cl. CPC G16H 40/40

(57) ABSTRACT

A disassembling accessory for disassembling a physiological signal monitoring device that includes a sensor kit having a through hole at a bottom portion thereof, and a transmitter removably coupled to the sensor kit. The disassembling accessory includes first and second housings movable relative each other between covering and open states. The first housing has an accommodating space. The second housing is connected to one side of the first housing, and has a pushing member. When the first and second housings are in the open state, the accommodating space opens toward the physiological signal monitoring device with the transmitter facing the accommodating space and being received in one of the first and second housings. When the first and second housings are in the covering state, the pushing member extends through the through hole to push the transmitter for disassembling the transmitter from the sensor kit.

### CROSS-REFERENCE TO RELATED APPLICATION

This application claims benefit of U.S. Provisional Patent Application No. 63/490,314, filed on Mar. 15, 2023, which is incorporated by reference herein in its entirety FIELD

The disclosure relates to a disassembling accessory, and more particularly to a disassembling accessory for disassembling a physiological signal monitoring device.

### BACKGROUND

Blood glucose control is critical to patients suffering from diabetes or to reducing complications caused thereby. Continuous glucose monitoring (CGM) is a popular method for tracking changes in glucose levels by taking glucose measurements of an individual at regular intervals.

A CGM system is mounted on a host, i.e., a patient suffering from diabetes, and usually includes a biosensor inserted beneath the host's skin for measuring a physiological signal corresponding to the blood glucose concentration level of the host, a transmitter for transceiving the physiological signal, and a mounting seat for interconnecting the biosensor and the transmitter and mounting on the host. Due to such intrusive nature of the biosensor, the host's body may be hypersensitive to the biosensor and in turn develops a severe allergic reaction, so the biosensor needs to be replaced on a weekly or bi-weekly basis. Given that the transmitter of the CGM system is more expensive than the biosensor and the mounting seat, frequent replacement of these electric components may also cause environmental waste.

### SUMMARY

Therefore, an object of the disclosure is to provide a disassembling accessory for disassembling a transmitter of a physiological signal monitoring device.

According to a first aspect of the disclosure, a disassembling accessory adapted for disassembling a physiological signal monitoring device is provided. The physiological signal monitoring device includes a sensor kit that has at least one through hole at a bottom portion thereof, and a transmitter that is removably coupled to the sensor kit. The disassembling accessory includes a first housing and a second housing. The first housing has an accommodating space.

The second housing is connected to one side of the first housing and has at least one pushing member. The first housing and the second housing are operable to move relative to each other between a covering state and an open state. When the first housing and the second housing are in the open state, the accommodating space is adapted to open toward the physiological signal monitoring device with the transmitter facing the accommodating space and being received in one of the first housing and the second housing. When the first housing and the second housing are in the covering state, the at least one pushing member is operable to move in a first direction such that the at least one pushing member is adapted to extend through the at least one through hole to push the transmitter for disassembling the transmitter from the sensor kit.

According to a second aspect of the disclosure, a method for disassembling a physiological signal monitoring device is provided. The physiological signal monitoring device includes a sensor kit that is formed with at least one opening at a bottom portion thereof, and a transmitter that is removably mounted to and covering the sensor kit. The method includes steps of:
providing a disassembling accessory as previously described according to the first aspect of the disclosure;
uncovering one of the first housing and the second housing so that the first housing and the second housing are in the open state;
placing the physiological signal monitoring device into one of the first housing and the second housing with the transmitter facing and corresponding to accommodating space;
covering one of first housing and the second housing so that the first housing and the second housing are in the covering state;
disassembling the transmitter from the sensor kit by driving the at least one pushing member to move in a first direction, to extend through the at least one opening of the bottom portion of the sensor kit, and to push the transmitter; and
retrieving the transmitter by removing the transmitter from the first housing.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is an exploded perspective view of a physiological signal monitoring device to be disassembled by a disassembling accessory of the present disclosure.

FIG. 2 is a sectional view of the physiological signal monitoring device.

FIG. 3 is a perspective view of a disassembling accessory of a first embodiment according to the present disclosure.

FIG. 4 is a partly exploded perspective view of the disassembling accessory of the first embodiment disposed in an open state.

FIG. 5 is an exploded perspective view of the disassembling accessory of the first embodiment

FIG. 6 is a perspective view of the physiological signal monitoring device being disposed in the disassembling accessory of the first embodiment when a first housing and a second housing are of the disassembling accessory are in the open state.

FIG. 7 is a sectional view of the disassembling accessory of the first embodiment of FIG. 6.

FIG. 8 is a sectional view of the disassembling accessory of the first embodiment.

FIG. 9 is a sectional view taken along the line IX-IX in FIG. 8.

FIG. 10 is a sectional view similar to FIG. 9, but illustrating a button of the disassembling accessory of the first embodiment being pressed to disassemble a transmitter from a sensor kit of the physiological signal monitoring device.

FIG. 11 is a sectional view similar to FIG. 10, but illustrating the transmitter being disengaged from the sensor kit.

FIG. 12 is a sectional view similar to FIG. 11, but illustrating disassembling of the transmitter being completed.

FIGS. 13(a) to 13(d) are perspective views of the disassembling accessory of the first embodiment, illustrating the physiological signal monitoring device being disposed in the disassembling accessory, being disassembled, and being removed from the disassembling accessory.

FIG. 14 is sectional view of a modification of the disassembling accessory of the first embodiment.

FIG. 15 is a perspective view of a disassembling accessory of a second embodiment according to the present disclosure.

FIG. 16 is a sectional view of the disassembling accessory of the second embodiment.

FIG. 17 is a sectional view of a modification of the disassembling accessory of the second embodiment.

FIG. 18 is a sectional view of a disassembling accessory of a third embodiment according to the present disclosure.

FIGS. 19(a) to 19(d) are perspective views of the disassembling accessory of the third embodiment, illustrating the physiological signal monitoring device being disposed in the disassembling accessory, being disassembled, and being removed from the disassembling accessory.

FIG. 20 is a perspective view of a disassembling accessory of a fourth embodiment according to the present disclosure.

FIG. 21 is a sectional view taken along the line XXI-XXI in FIG. 20.

FIG. 22 is a disassembling accessory of a fifth embodiment according to the present disclosure for disassembling the physiological signal monitoring device.

FIG. 23 is a sectional view taken along line XXIII-XXIII in FIG. 22.

FIG. 24 is an accessory box of the first embodiment according to the present disclosure for disassembling the physiological signal monitoring device.

FIG. 25 is a schematic perspective view of the accessory box of the first embodiment disposed in an open state.

FIG. 26 is a sectional view of the accessory box of the first embodiment.

FIG. 27 is a perspective view of an accessory box of a second embodiment according to the present disclosure.

FIG. 28 is a sectional view of an accessory box of a third embodiment according to the present disclosure.

FIG. 29 is a flow chart of a method for disassembling the physiological signal monitoring device to be implemented by the present disclosure.

### DETAILED DESCRIPTION

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

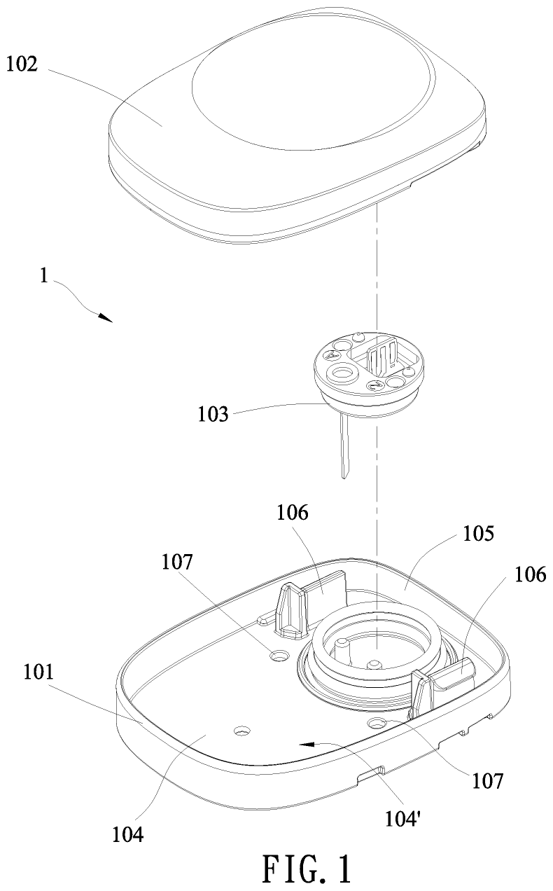

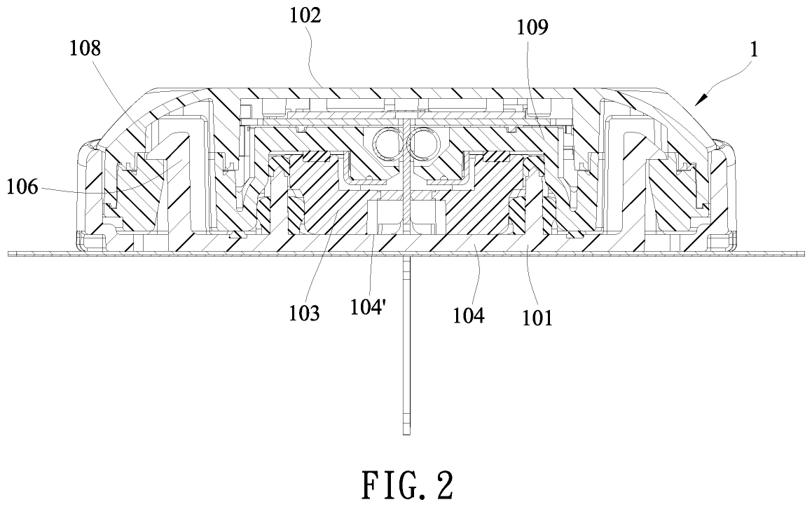

Referring to FIGS. 1 and 2, a physiological signal monitoring device 1 is shown. The physiological signal monitoring device 1 includes a bottom seat 101, a sensor 103 mounted to the bottom seat 101, and a transmitter 102 removably coupled to and covers on the bottom seat 101. The bottom seat 101 has a bottom plate 104 having a top surface 104′, a surrounding wall 105 extending upwardly from a periphery of the bottom plate 104, two first coupling structures 106 disposed on and protruding upwardly from the top surface 104′, and two through holes 107 formed through the bottom plate 104 and disposed respectively adjacent to the first coupling structures 106. The bottom plate 104 is disposed to be mounted on a skin surface of a host. The sensor 103 is for sensing an analyte of a host and for transmitting a physiological signal corresponding to the blood glucose concentration level of the host to the transmitter 102. The transmitter 102 is electrically connected to the sensor 103 for transceiving the physiological signal when covering on the bottom seat 101. In one embodiment, the bottom seat 101 and the sensor 103 are integrated as a sensor kit that is disposable, whereas the transmitter 102 may be reused.

Specifically, the transmitter 102 includes a bottom casing 109 and two second coupling structures 108. The second coupling structures 108 are disposed on the bottom casing 109. When the transmitter 102 covers the bottom seat 101, the second coupling structures 108 respectively and removably engage the first coupling structures 106 with the bottom casing 109 facing the top surface 104′ of the bottom plate 104. However, the quantities of the first coupling structures 106 and the second coupling structures 108 are not limited to any one of the embodiments of the present disclosure.

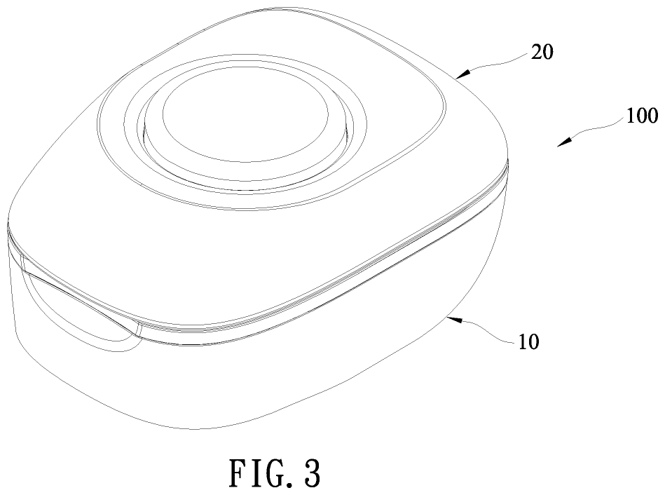

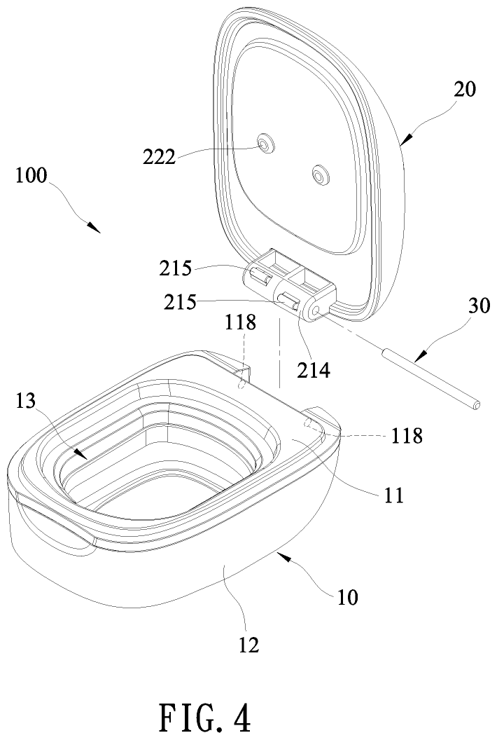

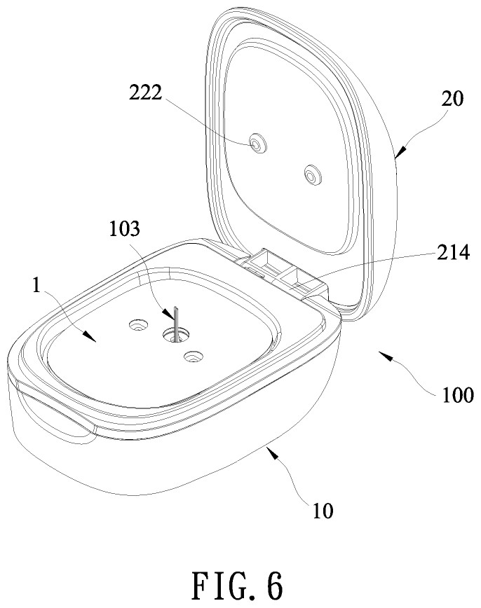

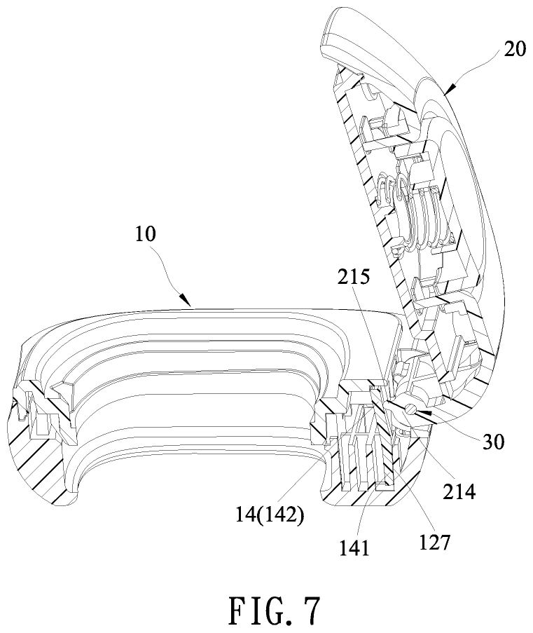

Referring to FIGS. 3 to 9, a dissembling accessory 100 of a first embodiment according to the present disclosure is shown. The dissembling accessory 100 is adapted for disassembling the physiological signal monitoring device 1 and includes a first housing 10 and a second housing 20 disposed above the first housing 10 in an up-down direction. The first housing 10 and the second housing 20 are operable to move relative to each other between a covering state and an open state. Specifically, the first housing 10 has an accommodating space 13, and the second housing 20 is connected to one side of the first housing 10, and is operable to move relative to the first housing 10 between the covering state (see FIG. 3) and the open state (see FIG. 6), where the second housing 20 covers and uncovers the first housing 10, respectively. The second housing 20 includes a button 23 and at least one pushing member 24 linked to and driven by the button 23 to move when the second housing 20 is in the cover state. As shown in FIGS. 6 and 7, when the second housing 20 is in the open state, the physiological signal monitoring device 1 is disposed in the accommodating space 13 with the transmitter 102 facing the accommodating space 13. Specifically, the physiological signal monitoring device 1 is disposed in the accommodating space 13 with the transmitter 102 facing a bottom of the accommodating space 13. In this embodiment, the disassembling accessory 100 includes a pivot shaft 30 that pivotably connects the second housing 20 to the first housing 10.

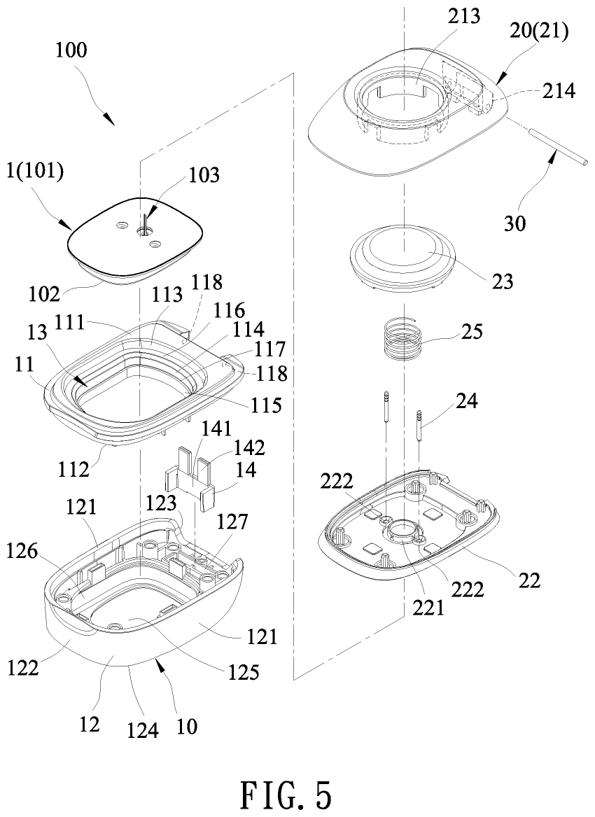

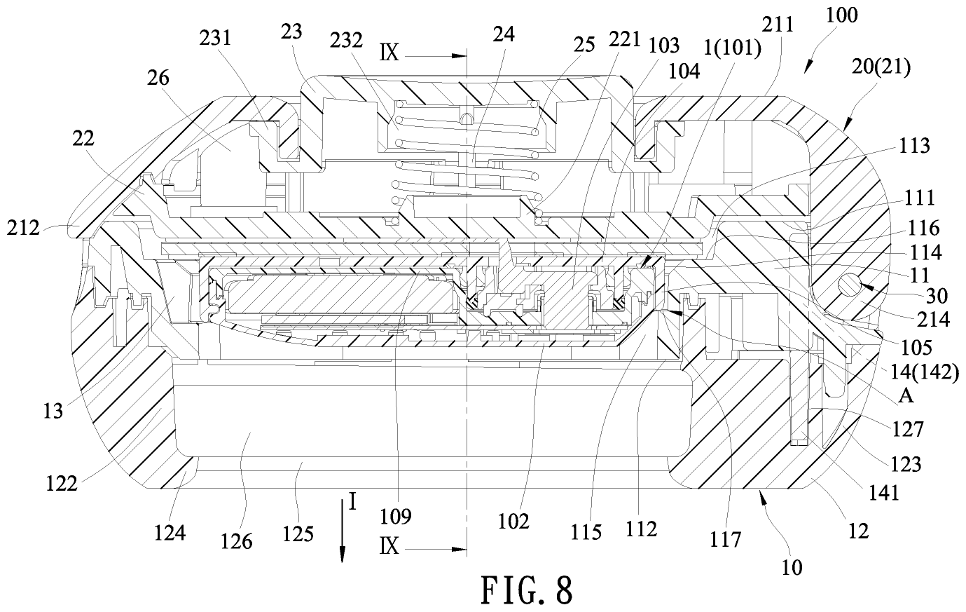

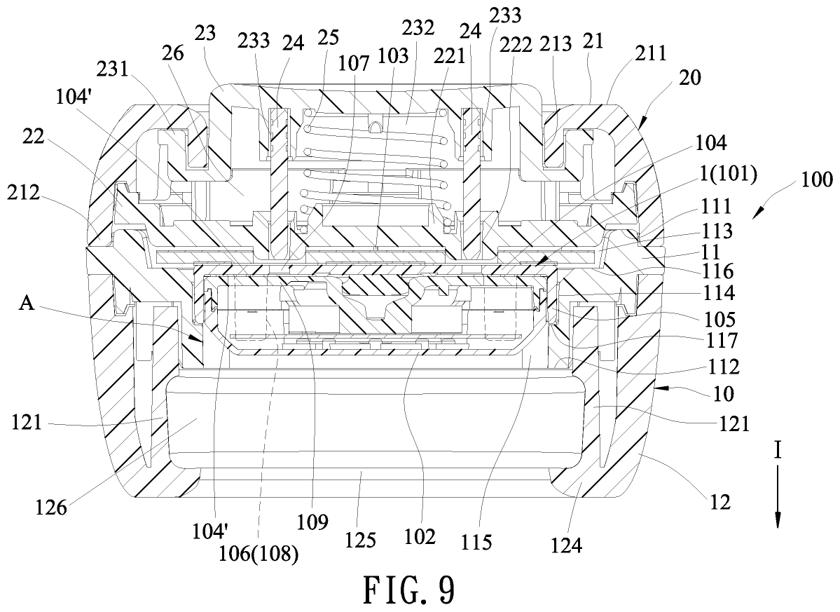

As shown in FIGS. 8 and 9, a sectional view of the disassembling accessory 100 of the first embodiment is shown. The first housing 10 includes a first housing body 11 and a second housing body 12 that is connected to the first housing body 11 and that cooperates with the first housing body 11 to define the accommodating space 13. As shown in FIG. 5, the first housing body 11 is an annular frame and has an upper surface 111, a lower surface 112 opposite to the upper surface 111, a recess portion 113 formed in the upper surface 111, a first receiving space 114 formed at a position opposite to the recess portion 113, and a through hole 115 formed in the lower surface 112. The first housing body 11 further has an upper shoulder surface 116 formed between the recess portion 113 and the first receiving space 114, a lower shoulder surface 117 formed between the first receiving space 114 and the lower surface 112, and a pair of pivot connecting holes 118 formed at a rear portion of the first housing body 11.

The second housing body 12 includes a pair of side walls 121, a front wall 122, a rear wall 123, and a transverse wall 124. The side walls 121 are spaced apart from each other. The front wall 122 is connected to the side walls 121. The rear wall 123 is opposite to the front wall 122 and is connected to the side walls 121. The transverse wall 124 is formed with a first opening 125 and is connected to the side walls 121, the front wall 122, and the rear wall 123. The side walls 121, the front wall 122, the rear wall 123, and the transverse wall 124 cooperate with one another to define a second receiving space 126 that is in spatial communication with the first receiving space 114, that is disposed under the first receiving space 114 in the up-down direction. Specifically, the accommodating space 13 has the first receiving space 114 and the second receiving space 126. As shown in FIGS. 8 and 9, the lower shoulder surface 117, the through hole 115, and the bottom surface 112 cooperate with one another to define a limiting structure. It should be noted that the limiting structure is formed in the first receiving space 114 and is for limiting the physiological signal monitoring device 1 therein when the physiological signal monitoring device 1 is disposed in the accommodating space 13 while facing a bottom of the accommodating space 13.

The second receiving space 126 is in spatial communication with external environment via the first opening 125. As depicted in FIG. 8, the first opening 125 has an opening size that is adapted to be greater than a maximum cross sectional area of the transmitter 102 or that is not smaller than a transverse wall area of the bottom casing 109 of the transmitter 102. The first receiving space 114 and the second receiving space 126 are arranged in a first direction and are proximate to each other.

As shown in FIGS. 5 and 8 to 9, the second housing 20 includes a first lid 21 and a second lid 22 connected to the first lid 21 and cooperating with the first lid 21 to define an inner space 26. The button 23 is mounted on the first lid 21. It should be noted that the number of the at least one pushing member 24 is two in this embodiment and the pushing members 24 are driven by the button 23 to move. In this embodiment, the second housing 20 further includes a biasing member 25 disposed in the inner space 26, disposed between and abutting against the second lid 22 and the button 23, and biasing the button 23 away from the second lid 22.

The first lid 21 is formed with a central hole 213 for mounting the button 23 therein, and has an upper portion 211, a lower portion 212 opposite to the upper portion 211, and a lug portion 214 formed at a rear of the first lid 21. The pivot shaft 30 extends through the lug portion 214 and the pivot connecting holes 118 to pivotably connect the second housing 20 and the first housing 10.

The second lid 22 includes a positioning ring 221 aligned with the central hole 213 in the first direction, and is formed with two sliding holes 222 that are disposed at two opposite sides of the positioning ring 221.

The button 23 is mounted in the central hole 213 of the first lid 21, is movable relative to the first lid 21, includes a rim portion 231 disposed in the inner space 26 and engaging the first lid 21, and is formed with a positioning hole 232 aligned with the positioning ring 221 in the first direction, and two insertion holes 233 aligned respectively with the sliding holes 222 and respectively receiving the pushing members 24 therein. That is to say, each of the pushing members 24 extends into the respective one of the insertion holes 233 of the button 23 and one of the sliding holes 222 that is aligned with the respective one of the insertion holes 233.

The pushing members 24 are disposed in the inner space 26 and are driven by the button 23 to move in the first direction and extend outwardly of the second lid 22. In this embodiment, the pushing members 24 are configured as two pillars, and extend through and are slidable along the sliding holes 222, respectively.

The biasing member 25 provides a resilient force for biasing the button 23 in a direction away from the second lid 22. In this embodiment, the biasing member 25 is a compression spring, has two opposite ends abutting respectively against the second lid 22 and the button 23, is disposed in the positioning hole 232, and surrounds the positioning ring 221. That is to say, the biasing member 25 is disposed between the positioning ring 221 and the positioning hole 232.

As shown in FIGS. 5 and 7, the disassembling accessory 100 further includes a rotation limiting member 14 mounted to the first housing 10 at a position adjacent to the pivot shaft 30 and disposed for positioning the second housing 20 relative to the first housing 10 in the open state. Specifically, the second housing body 12 is formed with a slot 127 at the rear wall 123. The rotational member 14 includes a positioning segment 141 and a blocking segment 142 connected to the positioning segment 141 and extending toward the first housing body 11. The first lid 21 further includes an abutment block 215 (see FIG. 7) disposed adjacent to the lug portion 214 and the rotational member 14. When the second housing 20 is in the open state, the abutment block 215 abuts against the blocking segment 142, which thus prevents unintentional movement of the second housing 20 from the open state to the covering state.

FIG. 3 illustrates the second housing 20 being in the covering state.

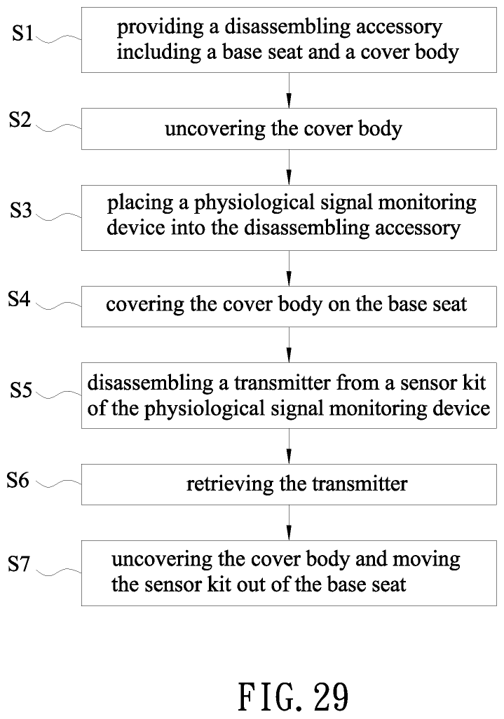

Referring to FIG. 29, a method for dissembling the physiological signal monitoring device 1 implemented by the disassemble accessory 100 is described in the following. The method includes the following steps S1 to S5.

In step S1, the dissembling accessory 100 that is shown in FIGS. 1 to 9 and that includes the first housing 10 and the second housing 20 described above is provided.

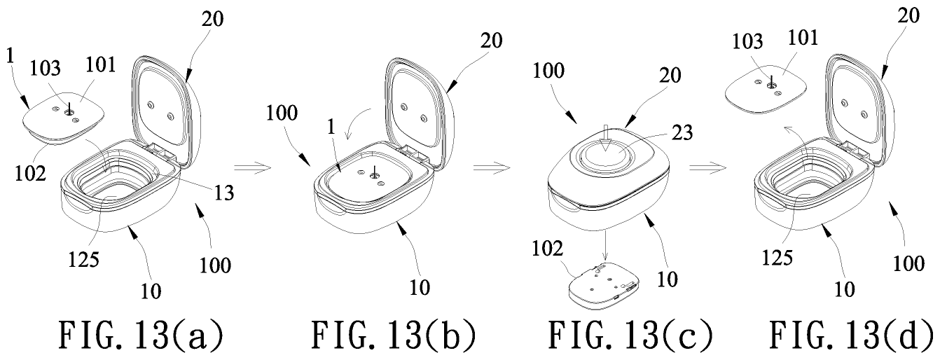

In step S2, as shown in FIGS. 6 and 13(a), the second housing 20 is uncovered (i.e., in the open state) relative to the first housing 10.

In step S3, as shown in FIGS. 6 and 13(b), the physiological signal monitoring device 1 is placed into the accommodating space 13 with the transmitter 102 facing the bottom of the accommodating space 13. Further referring to FIGS. 8 and 9, the limiting structure limits the sensor kit of the physiological signal monitoring device 1. Specifically, the surrounding wall 105 of the bottom seat 101 is disposed on and limited by the limiting structure, so the physiological signal monitoring device 1 is received in a portion of the accommodating space 13, i.e., the first receiving space 114. Upon the step of dissembling the transmitter 102 from the sensor kit, the transmitter 102 is moved to another portion of the accommodating space 13, i.e., the second receiving space 126.

In step S4, as shown in FIGS. 8 and 9, the second housing 20 covers the first housing 10 (i.e., in the covering state). It should be noted that, as depicted in FIG. 8, the sensor 103 has a portion that is bent when being covered by the second housing 20. When the second housing 20 is in the covering state, the button 23 is operable to drive the pushing members 24 to move in the first direction such that the pushing members 24 are adapted to respectively extend through the through holes 107 of the bottom seat 101 to push the transmitter 102 for disassembling the transmitter 102 from the first housing 10. Specifically, the button 23 is operated in the first direction and thus the pushing members 24 are moved in the first direction respectively through the through holes 107, but the present disclosure is not limited in this respect.

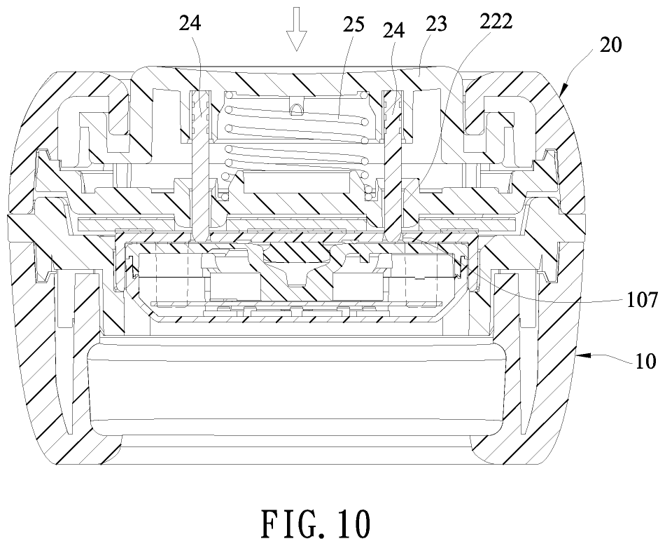

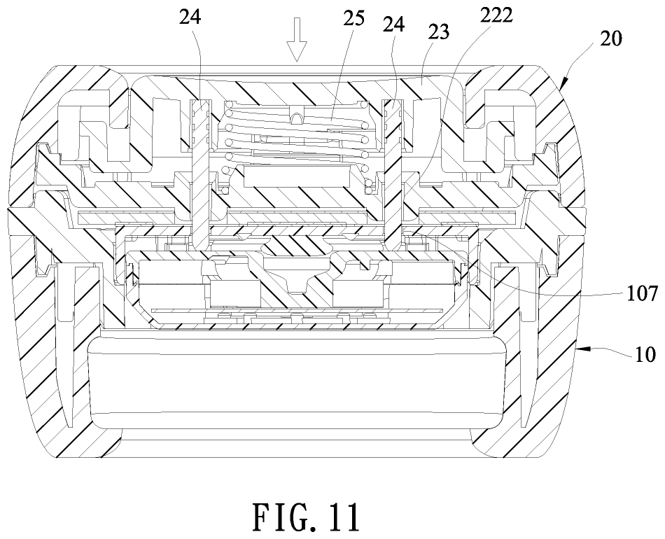

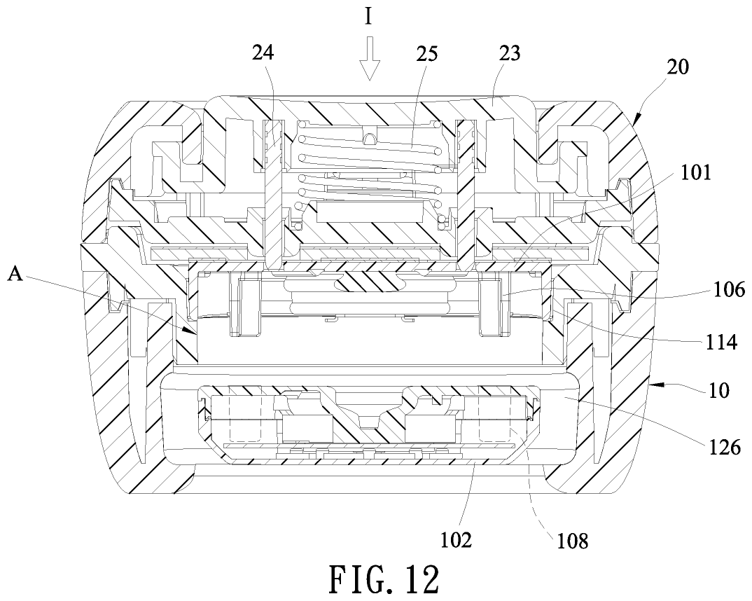

In step S5, as shown in FIGS. 10 to 12, and 13(c), the transmitter 102 is disassembled from the sensor kit. Specifically, the button 23 is pushed in the first direction to move the pushing members 24 in the first direction to respectively extend through the through holes 107 and to push the transmitter 102. At this time, the biasing member 25 is compressed to store a restoring force. Consequently, as shown in FIG. 12, the second coupling structures 108 disengage respectively from the first coupling structures 106 to thereby disassembling the transmitter 102 from the bottom seat 101. It should be noted that the transmitter 102 falls out of the first receiving space 114 and into the second receiving space 126 while the bottom seat 101 is retained on the limiting structure. After the button 23 is released, the restoring force provided by the biasing member 25 biases the button 23 away from the second lid 22 to its original position.

In step S6, the transmitter 102 is retrieved to be reused. As shown in FIG. 13(c), the transmitter 102 falls out of the first housing 10 through the first opening 125.

After the transmitter 102 is disassembled from the first housing 10, in step S7, the second housing 20 is uncovered (i.e., in the open state) and the sensor kit, i.e., the bottom seat 101 and the sensor 103, is movable out of the first housing 10 by an external force that is applied via the first opening 125.

Thus, when the sensor 103 is to be replaced, the transmitter 102 may be easily dissembled from the bottom seat 101 and may be reused. Since the transmitter 102 is more expansive than the bottom seat 101 and the sensor 103, the disassembling accessory 100 of the present disclosure lowers the cost for the host. Additionally, the present disclosure is beneficial to the environment, and corporate governance for social responsibility is achieved.

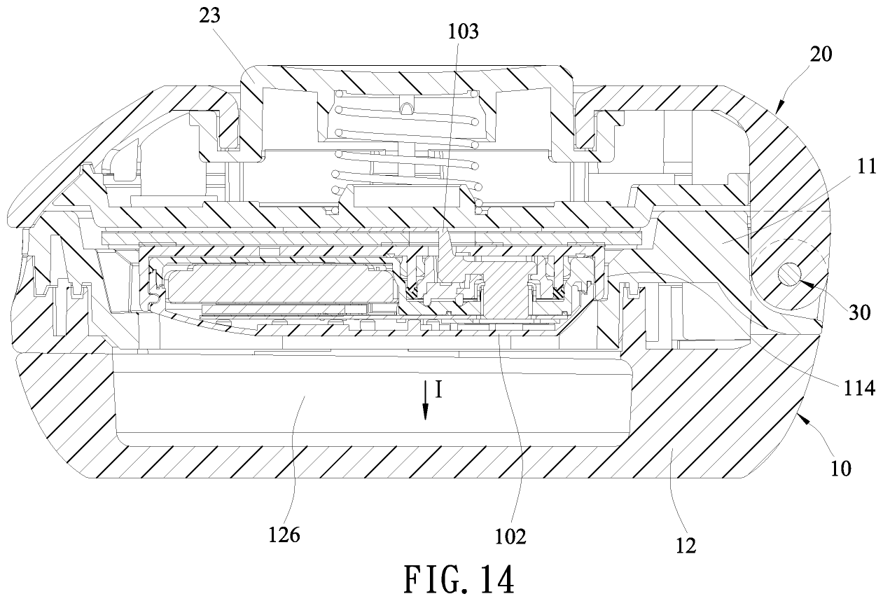

As shown in FIG. 14, a modification of the disassembling accessory 100 of the first embodiment is shown. In this modification, the first housing 10 includes a first housing body 11 defining the first receiving space 114, and a second housing body 12 defining the second receiving space 126 and connected pivotably to the first housing body 11 via the pivot shaft 30. The second housing body 12 is pivotable relative to the first housing body 11 from a limiting state to a non-limiting state, where removal of the transmitter 102 from the second receiving space 126 is permitted.

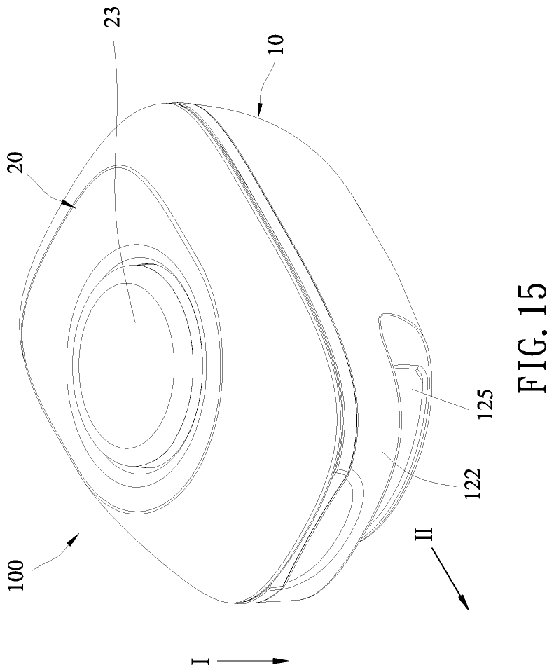

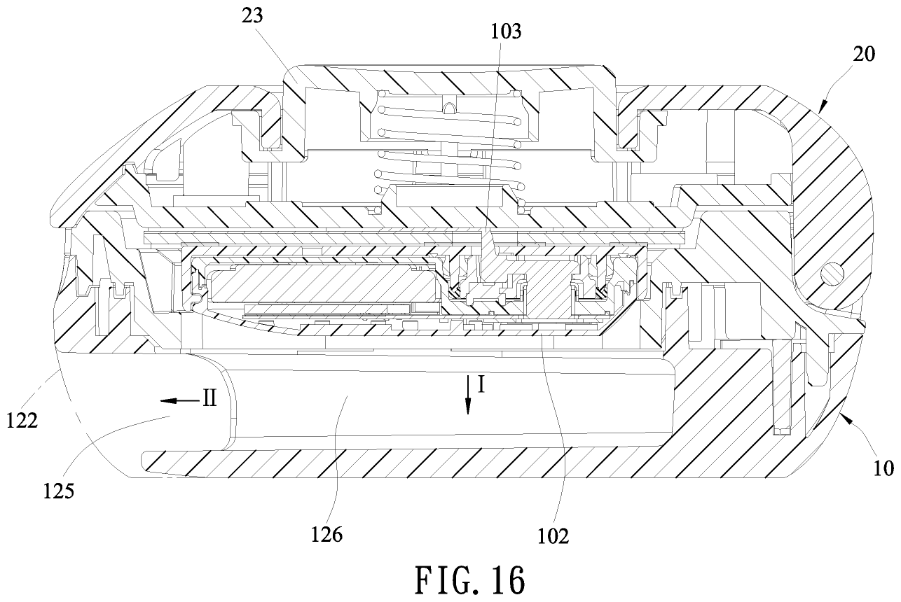

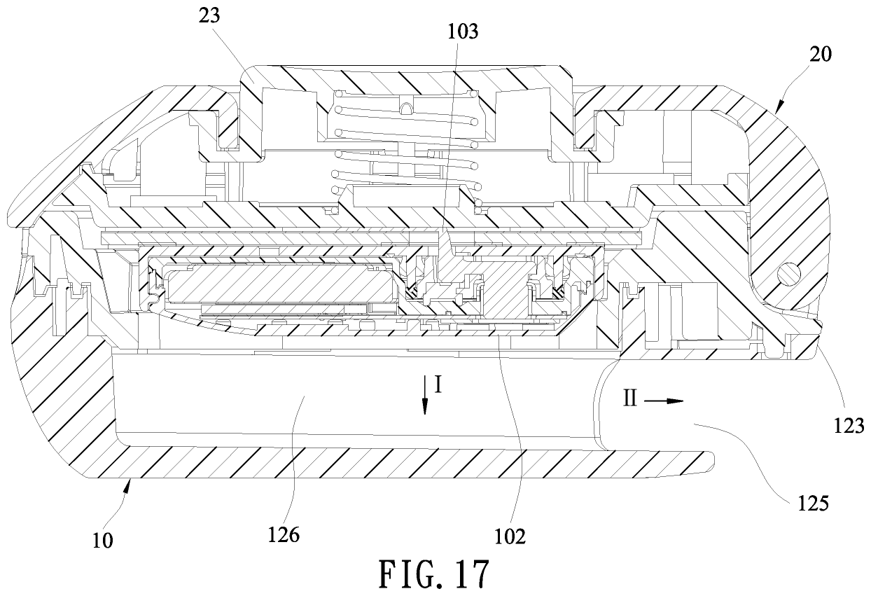

Referring to FIGS. 15 and 16, a disassembling accessory 100 of a second embodiment according to the present disclosure is shown. The second embodiment is similar to the first embodiment and also includes a first housing 10 and a second housing 20. The differences between the disassembling accessories 100 of the first embodiment and the second embodiment reside in the following. In the disassembling accessory 100 of the second embodiment, the first opening 125 is formed in the front wall 122 and is in spatial communication with the second receiving space 126. When the second housing 20 is in the covering state, the button 23 is operated to disassemble the transmitter 102 from the bottom seat 101 so that the transmitter 102 enters the second receiving space 126. Subsequently, the first opening 125 is adapted for passage of the transmitter 102 to exit from the second receiving space 126 to the external environment. In this way, the transmitter 102 may be removed outwardly of the first housing 10 via the first opening 125 in a second direction (II) transverse to the first direction by simply tilting the disassembling accessory 100. Thus, the transmitter 102 may be easily retrieved, and accidental fall of the transmitter 102 upon disassembling of the transmitter 102 from the bottom seat 101 may be prevented. As shown in FIG. 17, a modification of the disassembling accessory 100 of the second embodiment is shown. In this modification, the first opening 125 is formed in the rear wall 123 and is in spatial communication with the second receiving space 126.

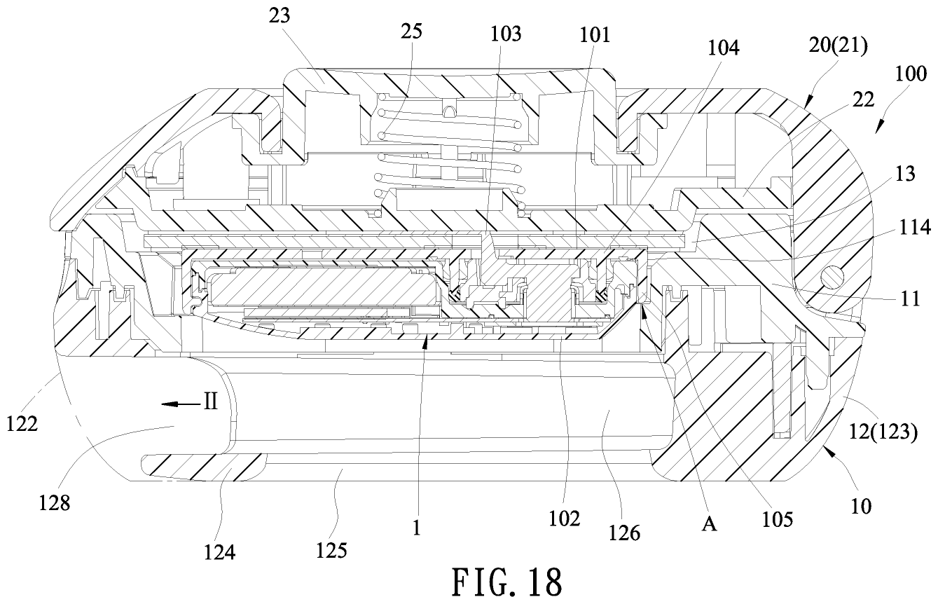

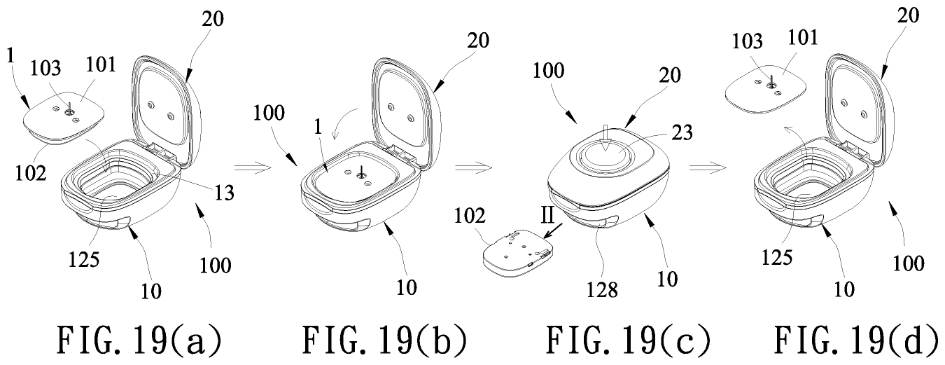

Referring to FIGS. 18 and 19, a disassembling accessory 100 of a third embodiment according to the present disclosure is similar to the disassembling accessory 100 of the first embodiment and also includes a first housing 10 and a second housing 20. It should be noted that the pushing members 24 are not visible in FIG. 16, and it may be appreciated that the pushing members 24 are shown in a sectional view similar to that shown in FIG. 9. The differences between the disassembling accessories 100 of the first embodiment and the third embodiment reside in the following. The first housing 10 of the disassembling accessory 100 of the third embodiment further has a second opening 128 that is formed in the front wall 122 and that is in spatial communication with the second receiving space 126 and the external environment, and the first opening 125 is formed in the transverse wall 124. In this embodiment, dimension of the first opening 125 is smaller than a maximum sectional area of the transmitter 102. The second opening 128 is adapted for passage of the transmitter 102 therethrough to exit from the second receiving space 126 to the external environment when the transmitter 102 is dissembled from the bottom seat 101. In this way, the transmitter 102 may be removed outwardly of the first housing 10 in the second direction (II) by simply tilting the disassembling accessory 100 to thereby retrieve and reuse the transmitter 102. After the transmitter 102 is dissembled from the bottom set 101, falls into the second receiving space 126, and exits from the second receiving space 126 via the second opening 128, the second housing 20 is uncovered (i.e., in the open state) and the bottom seat 101 and the sensor 103 are movable out of the first housing 10 by an external force, e.g., a user's finger, that is applied via the first opening 125. In this way, the sensor kit, i.e., the bottom seat 101 and the sensor 103, may be moved out of the first housing 10 via the first opening 125 and for replacement.

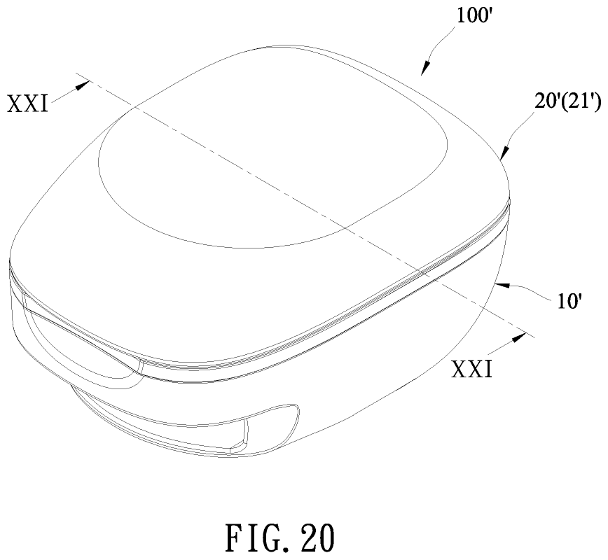

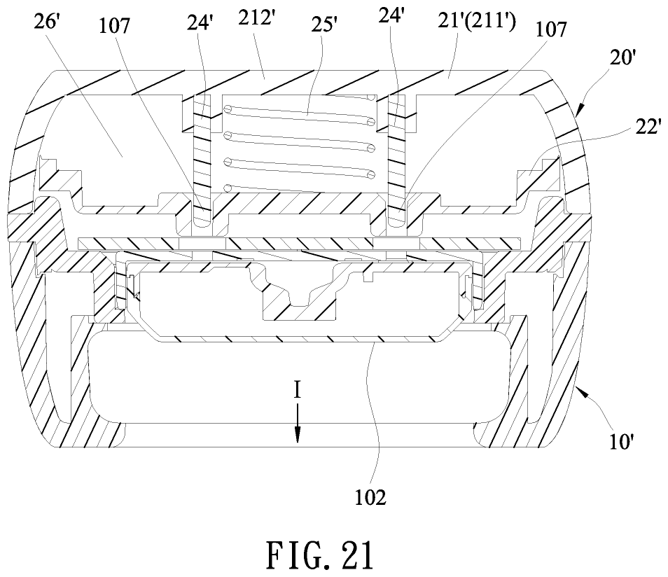

Referring to FIGS. 20 and 21, a disassembling accessory 100′ of a fourth embodiment according to the present disclosure is shown. FIG. 20 is a perspective view of the disassembling accessory 100′ and FIG. 21 is a sectional view of taken along the line XXI-XXI of FIG. 20. The fourth embodiment also includes a first housing 10′ and a second housing 20′.

As shown in FIG. 20, the differences between the disassembling accessories 100, 100′ of the first embodiment and the fourth embodiment reside in that the second housing 20′ of the fourth embodiment does not include the button 23 shown in FIG. 15. Specifically, in this embodiment, the second housing 20′ includes a first lid 21′, a second lid 22′ connected to the first lid 21′, a pair of pushing members 24′, and a biasing member 25′. The first lid 21′ includes a plate wall 211′ having at least one soft portion 212″ that is resiliently deformable and that is made of, e.g., silicon, through a two-material injection molding process. The soft portion 212″ is connected to and operable to drive the pushing members 24′ to move in the first direction such that the pushing members 24′ are adapted to extend respectively through the through hole 107 to push the transmitter 102 for disassembling the transmitter 102 from the sensor kit.

It should be noted that the biasing member 25′ is disposed in the inner space 26′, and is disposed between and abutting against the second lid 22′ and the first lid 21′ for restoring the soft portion 212′. Additionally, the biasing member 25′ facilitates the pushing members 24′ to stably extend through the through holes 107, respectively. In a case where the number of each of the through hole 107 and the pushing member 24′ is only one and the pushing member 24′ is disposed at a center of the first lid 21′, the biasing member 25′ surrounds the pushing member 24′ to enable stable movement of the pushing member 24 in the first direction through the through hole 107.

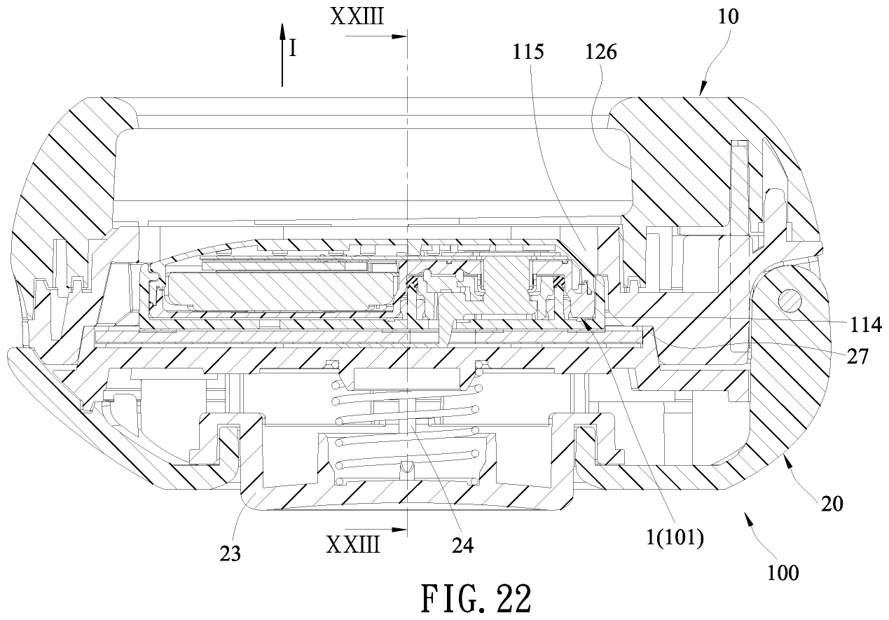

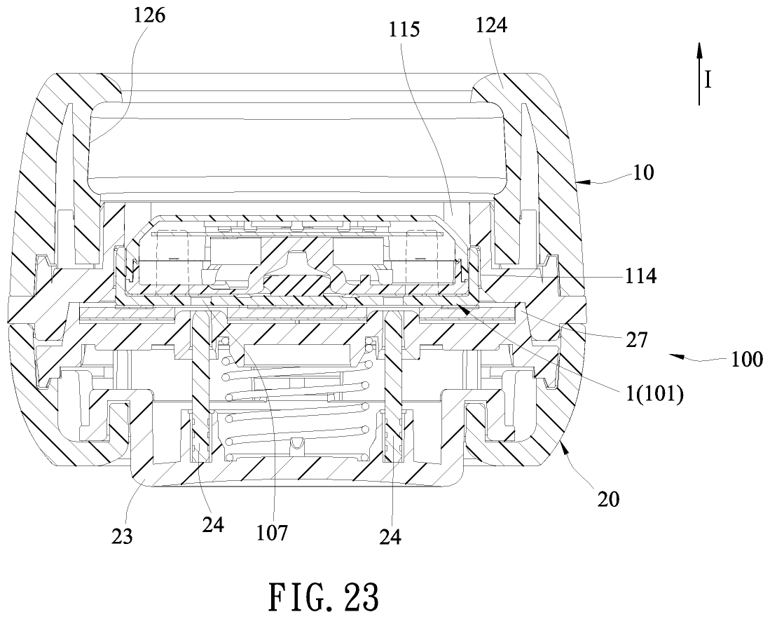

Referring to FIGS. 22 and 23, a disassembling accessory 100 of a fifth embodiment according to the present disclosure is shown. The difference between the fifth embodiment and the first embodiment resides in that the disassembling accessory 100 of the fifth embodiment have structures that are substantially upside down as compared to the structures of the first embodiment. In the fifth embodiment, the first housing 10 is disposed above the second housing 20 in the up-down direction. The first receiving space 114 is disposed under the second receiving space 126 in the up-down direction. The second housing 20 has a positioning set 27 adjoining the first receiving space 114 of the accommodating space 13. In this embodiment, the positioning set 27 is configured as an annular frame. It should be noted that the first receiving space 114 and the second receiving space 126 are still arranged in the first direction and are proximate to each other.

When the first housing 10 is in the open state, the positioning set 27 positions the physiological signal monitoring device 1 and is configured to align the pushing members 24 of the second housing 20 with the through holes 107, respectively. At this position, the button 23 faces downwardly in the up-down direction. When the first housing 10 is in the covering state relative to the second housing 20, the button 23 is operable to drive the pushing members 24 to move in the first direction such that the pushing members 24 are adapted to respectively extend through the through holes 107 to push the transmitter 102. It should be noted that the operation of the fifth embodiment is similar to the first embodiment, and the fifth embodiment possesses the advantages of the first embodiment.

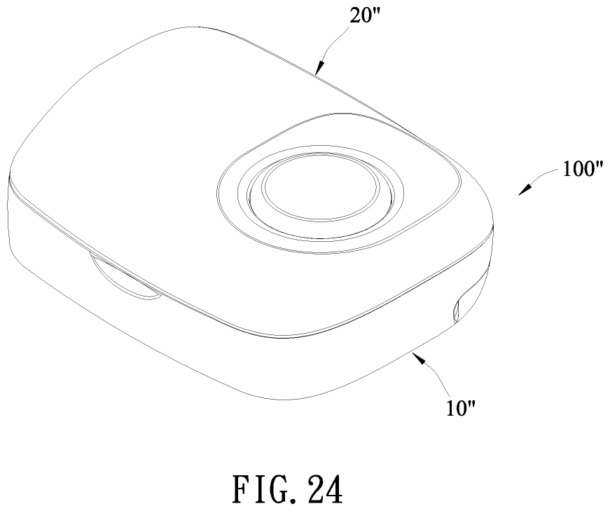

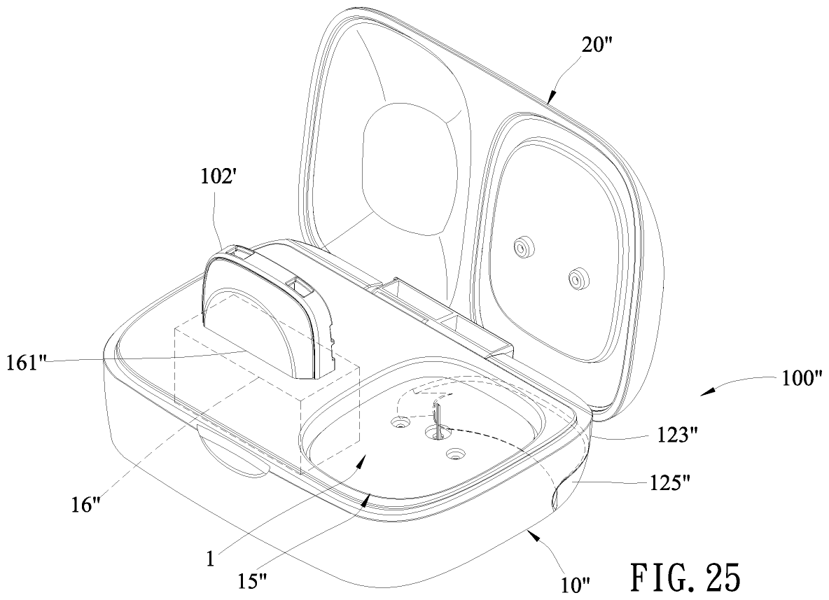

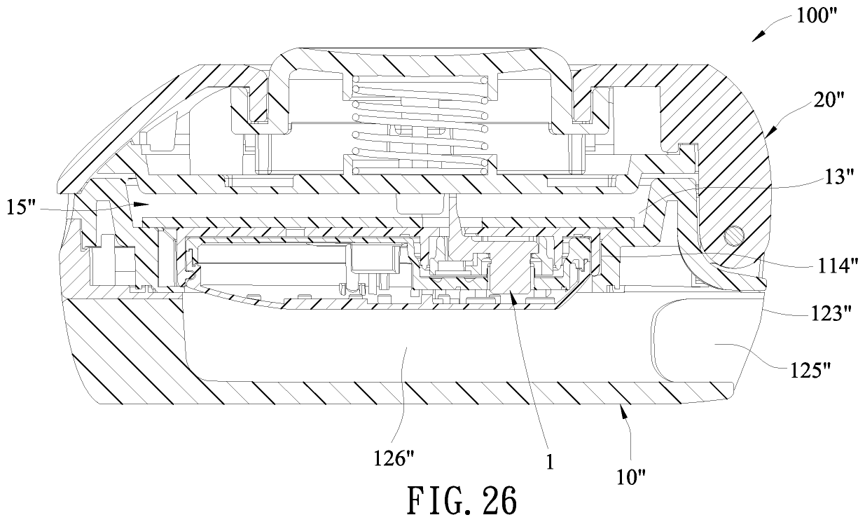

Referring to FIGS. 24 to 26, an accessory box 100″ of a first embodiment according to the present disclosure includes a first housing 10″ and a second housing 20″ pivotably connected to one side of the first housing 10″.

The first housing 10″ has a disassembling unit 15″ that has an accommodating space 13″, and a charging unit 16″ disposed adjacent to the dissembling unit 15″ and that is formed with a charging slot 161″. The accommodating space 13″ has a first receiving space 114″ and a second receiving space 126″ in spatial communication with the first receiving space 114″. The charging slot 161″ is adapted to receive the transmitter 102 that is disassembled from the sensor kit for charging a battery of the transmitter 102. The first opening 125″ is formed in the rear wall 123″ and is in spatial communication with the second receiving space 126″ and the external environment. The difference between the accessory box 100″ and the disassembling accessories 100, 100′ of previous embodiments merely resides in that the accessory box 100″ further includes the charging unit 16″ and the remaining features are substantially the same as those described above and are thus omitted for the sake of brevity.

When the second housing 20″ is in the open state, the physiological signal monitoring device 1 that is to be disassembled is placed in the accommodating space 13″, and the transmitter 102′ of a spare one of the physiological signal monitoring device 1 that is disassembled from the sensor kit of the spare one of the physiological signal monitoring device 1 is inserted in the charging slot 161″ such that a battery of the transmitter 102′ may be charged. In addition, the charging unit 16″ may include a charging port electrically connected to an external power source via a wire for charging the battery of the transmitter 102′. It should be noted that the charging port may be a wireless charging port electrically and wirelessly connected to the external power source.

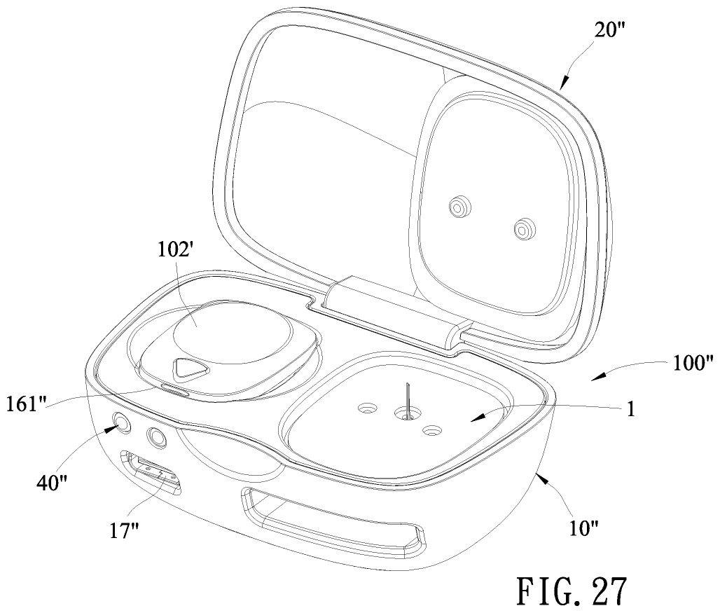

Referring to FIG. 27, an accessory box 100″ of a second embodiment according to the present disclosure is similar to the accessory box 100″ of the first embodiment and the difference therebetween resides in that the charging slot 161″ of the accessory box 100″ of the second embodiment has a flat configuration with a greater opening area than that of the charging slot 161″ of the accessory box 100″ of the first embodiment. Additionally, in this embodiment, the charging unit 16″ further includes a charging port 17″, e.g., type C, that is electrically connected to an external power source via a wire for charging the battery of the transmitter and a charging terminal (not shown) that is electrically connected to the charging port 17″ and the transmitter 102′ for charging the battery of the transmitter 102′. It can be appreciated that a wireless charging technique may be employed in other embodiments of this disclosure and the present disclosure is not limited in this respect.

Furthermore, the accessory box 100″ of the second embodiment further includes an indicating unit 40″ that is disposed on an outer surface of the first housing 10″, that is electrically connected to the charging unit 16″, and that is configured to indicate a charging progress of the transmitter 102. The indicator unit 40″ may include a light emitting diode (LED).

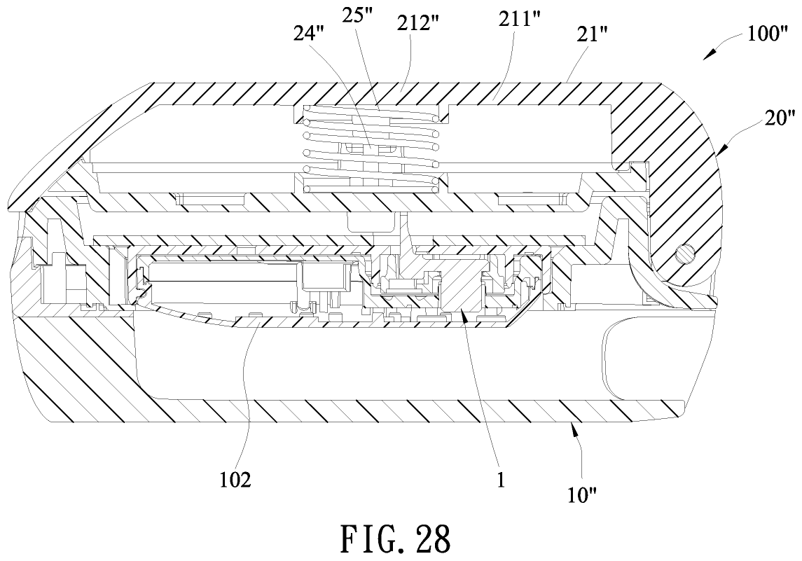

Referring to FIG. 28, an accessory box 100″ of a third embodiment according to the present disclosure is shown. In this embodiment, the second housing 20″ does not include the button 23 shown in FIG. 24, includes only one pushing member 24″, and the first lid 21″ includes a plate wall 211′ having at least one soft portion 212″ that is resiliently deformable and that is made of, e.g., silicon through a two-material injection molding process. The pushing member 24″ is surrounded by the biasing member 25″, is connected to and driven by the soft portion 212″ to move so the pushing member 24″ is moved in the first direction to push the transmitter 102 to be removed outwardly of the sensor kit.

In summary, each of the dissembling accessories 100, 100′ and the accessory boxes 100″ of the present disclosure has a simple structure, is easy to manufacture, and is convenient to disassemble the transmitter 102 from the sensor kit of the physiological signal monitoring device 1 for reuse. Thus, the cost for using a continuous glucose monitoring (CGM) system in a long term may be reduced, and the present disclosure is beneficial to the environment and corporate governance for social responsibility.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A disassembling accessory adapted for disassembling a physiological signal monitoring device, the physiological signal monitoring device including a sensor kit that has at least one through hole at a bottom portion thereof, and a transmitter that is removably coupled to the sensor kit, said disassembling accessory comprising:
a first housing that has an accommodating space; and
a second housing that is connected to one side of said first housing, and that has at least one pushing member, said first housing and said second housing being operable to move relative to each other between a covering state and an open state;
wherein, when said first housing and said second housing are in the open state, said accommodating space is adapted to open toward the physiological signal monitoring device with the transmitter facing said accommodating space and being received in one of said first housing and said second housing; and
wherein, when said first housing and said second housing are in the covering state, said at least one pushing member is operable to move in a first direction such that said at least one pushing member is adapted to extend through the at least one through hole to push the transmitter for disassembling the transmitter from the sensor kit.

2. The disassembling accessory as claimed in claim 1, the sensor kit including a bottom seat that is to be mounted on a skin surface of a host and that is formed with the at least one through hole, and a sensor that is mounted to the bottom seat and that is disposed between the transmitter and the bottom seat when the transmitter covers the bottom seat, wherein:
said at least one pushing member is adapted to extend through the at least one through hole of the bottom seat in the first direction to push the transmitter such that the transmitter is disassembled from the bottom seat and removed from said disassembling accessory in a second direction different from the first direction.

3. The disassembling accessory as claimed in claim 1, wherein:
said second housing is disposed above said first housing in an up-down direction;
said accommodating space of said first housing has a first receiving space and a second receiving space that is in spatial communication with said first receiving space, and that is disposed under said first receiving space in the up-down direction, said first receiving space and said second receiving space being arranged in the first direction;
when said first housing and said second housing are in the open state, said first receiving space is adapted to receive the physiological signal monitoring device therein with the transmitter facing said first receiving space; and
when said first housing and said second housing are in the covering state, said at least one pushing member is operable to move in the first direction such that said at least one pushing member is adapted to extend through the at least one through hole of the bottom portion of the sensor kit to push the transmitter so that the transmitter is disassembled from the sensor kit and enters said second receiving space.

4. The disassembling accessory as claimed in claim 3, wherein said first housing further has a first opening in spatial communication with said second receiving space and an external environment.

5. The disassembling accessory as claimed in claim 4, wherein:
said first housing includes a pair of side walls, a front wall connected to said side walls, a rear wall connected to said side walls and opposite to said front wall, and a transverse wall connected to said side walls, said front wall, and said rear wall;
said side walls, said front wall, said rear wall, and said transverse wall cooperate with each other to define said second receiving space; and
said first opening is formed in said transverse wall.

6. The disassembling accessory as claimed in claim 4, wherein:
said first housing includes a pair of side walls, a front wall connected to said side walls, a rear wall connected to said side walls and opposite to said front wall, and a transverse wall connected to said side walls, said front wall, and said rear wall;
said side walls, said front wall, said rear wall, and said transverse wall cooperatively define said second receiving space;
said first housing further has a second opening that is formed in one of said front wall and said rear wall and that is in spatial communication with said second receiving space and the external environment; and
said first opening is formed in one of said front wall and said rear wall.

7. The disassembling accessory as claimed in claim 6, wherein:
said first opening has an opening size that is adapted to be smaller than a transverse wall area of a transverse wall of the transmitter;
said second opening is adapted for passage of the transmitter therethrough to exit from said second receiving space to the external environment when the transmitter is disassembled from the sensor kit; and
when the transmitter is disassembled from the sensor kit and falls into said second receiving space, the sensor kit is movable out of said first housing by an external force that is applied via said first opening.

8. The disassembling accessory as claimed in claim 4, wherein:
said first housing includes a pair of side walls, a front wall connected to said side walls, a rear wall connected to said side walls and opposite to said front wall, and a transverse wall connected to said side walls, said front wall, and said rear wall;
said side walls, said front wall, said rear wall, and said transverse wall cooperatively define said second receiving space; and
said first opening is formed in one of said front wall and said rear wall.

9. The disassembling accessory as claimed in claim 3, wherein:
said first housing further has a limiting structure; and
said first receiving space is adapted to receive the physiological signal monitoring device therein with the transmitter facing said first receiving space, said limiting structure being adapted to limit the sensor kit of the physiological signal monitoring device in said first receiving space, and to consequently position the transmitter to be adjacent to said second receiving space.

10. The disassembling accessory as claimed in claim 3, further comprising a pivot shaft, wherein said first housing further includes
a first housing body defining said first receiving space, and
a second housing body defining said second receiving space, and connected pivotably to said first housing body via said pivot shaft; and
said second housing body is pivotable relative to said first housing body from a limiting state to a non-limiting state where removal of the transmitter from said second receiving space is permitted.

11. The disassembling accessory as claimed in claim 1, further comprising a pivot shaft, said second housing being pivotably connected to said first housing via said pivot shaft.

12. The disassembling accessory as claimed in claim 11, further comprising a rotation limiting member mounted to said first housing at a position that is adjacent to said pivot shaft and disposed for positioning said first housing and said second housing in the open state.

13. The disassembling accessory as claimed in claim 1, wherein:
said first housing is disposed above said second housing in an up-down direction;
when said first housing and said second housing are in the open state, said accommodating space is adapted to open toward the physiological signal monitoring device with the transmitter facing said accommodating space and being received in said second housing; and
when said first housing and said second housing are in the covering state, said at least one pushing member is operable to move in the first direction such that said at least one pushing member is adapted to extend through the at least one through hole to push the transmitter so that the transmitter is disassembled from the sensor kit and enters said accommodating space.

14. The disassembling accessory as claimed in claim 1, wherein:
said second housing has a positioning set adjoining said accommodating space and for positioning the physiological signal monitoring device within said disassembling accessory, and configured to align said at least one pushing member of said second housing with the at least one through hole of the bottom portion of the sensor kit.

15. The disassembling accessory as claimed in claim 1, wherein:
said cover body has a first lid, a second lid connected to said first lid, and an inner space defined by said first lid and said second lid;
a button is mounted on said first lid;
said second housing further includes a biasing member disposed in said inner space, and disposed between and abutting against said second lid and said button for biasing said button in a direction away from said second lid; and
said at least one pushing member is disposed in said inner space and is driven by said button to move, such that said at least one pushing member is driven to extend outwardly of said second lid.

16. The disassembling accessory as claimed in claim 1, wherein said second housing includes a plate wall having at least one soft portion that is operable to drive said at least one pushing member to move in the first direction such that said at least one pushing member is adapted to extend through the through hole to push the transmitter for disassembling the transmitter from the sensor kit.

17. The disassembling accessory as claimed in claim 16, wherein:
said second housing has a first lid, a second lid connected to said first lid, and an inner space defined by said first lid and said second lid;
said second housing further has
a soft portion formed on said first lid and being resiliently deformable, and
a biasing member disposed in said inner space, and disposed between and abutting against said first lid and said second lid for restoring said soft portion; and
said at least one pushing member is disposed in said inner space and is driven by said soft portion to move in the first direction, such that said at least one pushing member is adapted to extend outwardly of said second lid through the at least one through hole to push the transmitter for disassembling the transmitter from the sensor kit.

18. A method for disassembling a physiological signal monitoring device, the physiological signal monitoring device including a sensor kit that is formed with at least one through hole at a bottom portion thereof, and a transmitter that is removably coupled to the sensor kit, the method comprising steps of:
providing a disassembling accessory as claimed in claim 1;
uncovering one of the first housing and the second housing so that the first housing and the second housing are in the open state;
placing the physiological signal monitoring device into one of the first housing and the second housing with the transmitter facing and corresponding to the accommodating space;
covering one of the first housing and the second housing so that the first housing and the second housing are in the covering state;
disassembling the transmitter from the sensor kit by driving the at least one pushing member to move in a first direction, to extend through the at least one through hole of the bottom portion of the sensor kit, and to push the transmitter; and
retrieving the transmitter by removing the transmitter from the first housing.

19. The method as claimed in claim 18, the second housing further including a button that is operable to drive the pushing member to move, wherein the step of disassembling the transmitter from the sensor kit further includes:
pushing the button in the first direction to move the at least one pushing member in the first direction, to extend through the at least one through hole, and to push the transmitter.

20. The method as claimed in claim 18, the second housing of the disassembling accessory having a soft portion that is operable to drive the at least one pushing member to move, wherein the step of disassembling the transmitter from the sensor kit further includes:
pressing the soft portion to drive the at least one pushing member to move in the first direction such that the at least one pushing member extends through the through hole to push the transmitter.
